(19) United States
(12) Patent Application Publication
(10) Pub. No.: US 2012/0034203 A1
(43) Pub. Date: Feb. 9, 2012

(54) Methods for Killing or Inhibiting Growth of Mycobacteria

(75) Inventors: Rikke Festersen, Herlev (DK); Morten Gjermansen, Greve (DK)

(73) Assignee: NOVOZYMES A/S, Bagsvaerd (DK)

(21) Appl. No.: 13/259,826

(22) PCT Filed: Apr. 22, 2010
(86) PCT No.: PCT/EP2010/055339

§ 371 Date: Sep. 23, 2011

(30) Foreign Application Priority Data
Apr. 22, 2009 (EP) 09158526.5

(51) Int. Cl. A01N 63/00; A01P 1/00

(52) U.S. Cl. 424/94.4

(57) ABSTRACT

The present invention provides a method for killing or inhibiting growth of Mycobacteria, by contacting the Mycobacteria with a haloperoxidase, hydrogen peroxide, chloride ions and/or bromide ions, and ammonium ions.

### REFERENCE TO A SEQUENCE LISTING

This application contains a Sequence Listing in computer readable form. The computer readable form is incorporated herein by reference.

### FIELD OF THE INVENTION

The present invention relates to enzymatic methods for killing or inhibiting growth of Mycobacteria, and for disinfecting or sterilizing medical devices and equipment.

### BACKGROUND

Most vegetative cells of pathogenic bacteria are killed or inactivated within minutes at 70 degrees Celsius; however, some pathogenic bacteria, such as most Mycobacteria, are much more difficult to inactivate. As an alternative to heat treatment, disinfection may also be achieved by means of a chemical treatment, such as a glutaraldehyde treatment or a peracetic acid treatment. However, many modern medical devices, such as endoscopes and anaesthetic equipment, include complicated combinations of various sensitive materials and/or electronic appliances. Such medical devices are often sensitive to high temperatures and chemical treatment and often have a reduced service life when being repeatedly exposed to disinfection steps of the above described types. Accordingly, it is desirable to use a method for disinfection of medical equipment which employs lower temperatures and mild conditions, while retaining the Mycobacteria inactivating capabilities.

The present invention provides an improved method for killing or inhibiting growth of Mycobacteria, which is more gentle on sensitive medical equipment than traditional methods.

### SUMMARY

The present invention provides a method for killing or inhibiting growth of Mycobacteria, comprising contacting the Mycobacteria with a haloperoxidase, a source of hydrogen peroxide, chloride ions and/or bromide ions, and ammonium ions.

### DETAILED DESCRIPTION

### Haloperoxidases and Compounds Exhibiting Haloperoxidase Activity

The haloperoxidases suitable for being incorporated in the method of the invention include chloroperoxidases, bromoperoxidases and compounds exhibiting chloroperoxidase or bromoperoxidase activity. Haloperoxidases form a class of enzymes, which are capable of oxidizing halides (Cl—, Br—, I—) in the presence of hydrogen peroxide or a hydrogen peroxide generating system to the corresponding hypohalous acids.

Haloperoxidases are classified according to their specificity for halide ions. Chloroperoxidases (E.C. 1.11.1.10) catalyze formation of hypochlorite from chloride ions, hypobromite from bromide ions and hypoiodite from iodide ions; and bromoperoxidases catalyze formation of hypobromite from bromide ions and hypoiodite from iodide ions. Hypoiodite, however, undergoes spontaneous disproportionation to iodine and thus iodine is the observed product. These hypohalite compounds may subsequently react with other compounds forming halogenated compounds.

In a preferred embodiment, the haloperoxidase of the invention is a chloroperoxidase.

Haloperoxidases have been isolated from various organisms: mammals, marine animals, plants, algae, lichen, fungi and bacteria. It is generally accepted that haloperoxidases are the enzymes responsible for the formation of halogenated compounds in nature, although other enzymes may be involved.

Haloperoxidases have been isolated from many different fungi, in particular from the fungus group dematiaceous hyphomycetes, such as Caldariomyces, e.g., C. fumago, Alternaria, Curvularia, e.g., C. verruculosa and C. inaequalis, Drechslera, Ulocladium and Botrytis.

Haloperoxidases have also been isolated from bacteria such as Pseudomonas, e.g., P. pyrrocinia and Streptomyces, e.g., S. aureofaciens.

In a preferred embodiment, the haloperoxidase is a vanadium haloperoxidase (i.e. a vanadium or vanadate containing haloperoxidase) derivable from Curvularia sp., in particular Curvularia verruculosa or Curvularia inaequalis, such as C. inaequalis CBS 102.42 as described in WO 95/27046, e.g. a vanadium haloperoxidase encoded by the DNA sequence of WO 95/27046, FIG. 2 all incorporated by reference; or C. verruculosa CBS 147.63 or C. verruculosa CBS 444.70 as described in WO 97/04102. Preferably, the amino acid sequence of the haloperoxidase has at least 90% identity, preferably 95% identity to the amino acid sequence of a haloperoxidase obtainable from Curvularia verruculosa (see e.g. SEQ ID NO:2 in WO 97/04102; also shown as SEQ ID NO:1 in the present application/sequence listing) or Curvularia inequalis (e.g. the mature amino acid sequence encoded by the DNA sequence in FIG. 2 of WO 95/27046; also shown as SEQ ID NO:2 in the present application/sequence listing).

In another preferred embodiment the haloperoxidase is a vanadium containing haloperoxidase; in particular a vanadium chloroperoxidase. The vanadium chloroperoxidase may be derivable from Drechslera hartlebii as described in WO 01/79459, Dendryphiella salina as described in WO 01/79458, Phaeotrichoconis crotalarie as described in WO 01/79461, or Geniculosporium sp. as described in WO 01/79460. The vanadium haloperoxidase is more preferably derivable from Drechslera hartlebii (DSM 13444), Dendryphiella salina (DSM 13443), Phaeotrichoconis crotalarie (DSM 13441) or Geniculosporium sp. (DSM 13442).

The concentration of the haloperoxidase is typically in the range of 0.01-100 ppm enzyme protein, preferably 0.05-50 ppm enzyme protein, more preferably 1-40 ppm enzyme protein, more preferably 0.1-20 ppm enzyme protein, and most preferably 0.5-10 ppm enzyme protein.

In an embodiment, the concentration of the haloperoxidase is typically in the range of 5-50 ppm enzyme protein, preferably 5-40 ppm enzyme protein, more preferably 8-32 ppm enzyme protein.

### Determination of Haloperoxidase Activity

An assay for determining haloperoxidase activity may be carried out by mixing 100 μL of haloperoxidase sample (about 0.2 μg/mL) and 100 μL of 0.3 M sodium phosphate pH 7 buffer—0.5 M potassium bromide—0.008% phenol red, adding the solution to 10 μL of 0.3% H₂O₂, and measuring the absorption at 595 nm as a function of time.

Another assay using monochlorodimedone (Sigma M4632, ε=20000 M⁻¹ cm⁻¹ at 290 nm) as a substrate may be carried out by measuring the decrease in absorption at 290 nm as a function of time. The assay is done in an aqueous solution of 0.1 M sodium phosphate or 0.1 M sodium acetate, 50 μM monochlorodimedone, 10 mM KBr/KCl, 1 mM H₂O₂ and about 1 μg/mL haloperoxidase. One haloperoxidase unit (HU) is defined as 1 micromol of monochlorodimedone chlorinated or brominated per minute at pH 5 and 30° C.

### Hydrogen Peroxide

The hydrogen peroxide required by the haloperoxidase may be provided as an aqueous solution of hydrogen peroxide or a hydrogen peroxide precursor for in situ production of hydrogen peroxide. Any solid entity which liberates upon dissolution a peroxide which is useable by haloperoxidase can serve as a source of hydrogen peroxide. Compounds which yield hydrogen peroxide upon dissolution in water or an appropriate aqueous based medium include but are not limited to metal peroxides, percarbonates, persulphates, perphosphates, peroxyacids, alkyperoxides, acylperoxides, peroxyesters, urea peroxide, perborates and peroxycarboxylic acids or salts thereof.

Another source of hydrogen peroxide is a hydrogen peroxide generating enzyme system, such as an oxidase together with a substrate for the oxidase. Examples of combinations of oxidase and substrate comprise, but are not limited to, amino acid oxidase (see e.g. U.S. Pat. No. 6,248,575) and a suitable amino acid, glucose oxidase (see e.g. WO 95/29996) and glucose, lactate oxidase and lactate, galactose oxidase (see e.g. WO 00/50606) and galactose, and aldose oxidase (see e.g. WO 99/31990) and a suitable aldose.

By studying EC 1.1.3._, EC 1.2.3._, EC 1.4.3._, and EC 1.5.3._ or similar classes (under the International Union of Biochemistry), other examples of such combinations of oxidases and substrates are easily recognized by one skilled in the art.

Hydrogen peroxide or a source of hydrogen peroxide may be added at the beginning of or during the process, e.g., typically in an amount corresponding to levels of from 0.001 mM to 25 mM, preferably to levels of from 0.005 mM to 5 mM, and particularly to levels of from 0.01 to 1 mM hydrogen peroxide. Hydrogen peroxide may also be used in an amount corresponding to levels of from 0.1 mM to 25 mM, preferably to levels of from 0.5 mM to 15 mM, more preferably to levels of from 1 mM to 10 mM, and most preferably to levels of from 2 mM to 8 mM hydrogen peroxide.

### Chloride and Bromide Ions

According to the invention, the chloride and/or bromide ions (Cl⁻ and/or Br⁻) needed for the reaction with the haloperoxidase may be provided in many different ways, such as by adding salts of chloride and/or bromide. In a preferred embodiment the salts of chloride and bromide are sodium chloride (NaCl), sodium bromide (NaBr), potassium chloride (KCl), potassium bromide (KBr), ammonium chloride (NH₄Cl) or ammonium bromide (NH₄Br); or mixtures thereof.

In an embodiment, the chloride and/or bromide ions are limited to only chloride ions (Cl⁻) or bromide ions (Br⁻). In another embodiment, the chloride and/or bromide ions are limited to only chloride ions (Cl⁻) and bromide ions (Br⁻). The chloride ions may be provided by adding a salt of chloride to an aqueous solution. The salt of chloride may be sodium chloride, potassium chloride or ammonium chloride; or a mixture thereof. The bromide ions may be provided by adding a salt of bromide to an aqueous solution. The salt of bromide may be sodium bromide, potassium bromide or ammonium bromide; or a mixture thereof.

The concentration of each of chloride and bromide ions are typically in the range of from 0.01 mM to 1000 mM, preferably in the range of from 0.05 mM to 500 mM, more preferably in the range of from 0.1 mM to 100 mM, most preferably in the range of from 0.1 mM to 50 mM, and in particular in the range of from 1 mM to 25 mM. The concentration of chloride ions is independent of the concentration of bromide ions; and vice versa.

In an embodiment, the molar concentration of each of chloride and bromide ions is at least two times higher, preferably at least four times higher, more preferably at least six times higher, most preferably at least eight times higher, and in particular at least ten times higher than the concentration of ammonium ions.

### Ammonium Ions

The ammonium ions (NH₄⁺) needed to kill or inhibit growth of Mycobacteria according to the methods of the invention may be provided in many different ways, such as by adding a salt of ammonium. In a preferred embodiment the ammonium salt is ammonium sulphate ((NH₄)₂SO₄), ammonium carbonate ((NH₄)₂CO₃), ammonium chloride (NH₄Cl), ammonium bromide (NH₄Br), or ammonium iodide (NH₄I); or a mixture thereof.

The concentration of ammonium ions is typically in the range of from 0.01 mM to 1000 mM, preferably in the range of from 0.05 mM to 500 mM, more preferably in the range of from 0.1 mM to 100 mM, most preferably in the range of from 0.1 mM to 50 mM, and in particular in the range of from 1 mM to 25 mM.

### Mycobacteria

The Mycobacteria which are killed or inactivated with a haloperoxidase, hydrogen peroxide, chloride ions and/or bromide ions, and ammonium ions according to the invention, may be any Mycobacterium sp., such as species from the Mycobacterium tuberculosis complex (MTBC).

In an embodiment, the Mycobacteria of the invention are capable of causing tuberculosis.

In another embodiment, the Mycobacteria are selected from the group consisting of M. tuberculosis, M. bovis, M. bovis BCG, M. africanum, M. microti, M. canetti, M. caprae and M. pinnipedii.

In a prefered embodiment, the Mycobacteria according to the invention are Mycobacterium tuberculosis or Mycobacterium bovis cells.

### Surfactants

The method of the invention may include application of a surfactant (for example, as part of a detergent formulation or as a wetting agent). Surfactants suitable for being applied may be non-ionic (including semi-polar), anionic, cationic and/or zwitterionic; preferably the surfactant is anionic (such as linear alkylbenzenesulfonate, alpha-olefinsulfonate, alkyl sulfate (fatty alcohol sulfate), alcohol ethoxysulfate, secondary alkanesulfonate, alpha-sulfo fatty acid methyl ester, alkyl- or alkenylsuccinic acid or soap) or non-ionic (such as alcohol ethoxylate, nonylphenol ethoxylate, alkylpolyglycoside, alkyldimethylamineoxide, ethoxylated fatty acid monoethanolamide, fatty acid monoethanolamide, polyhydroxy alkyl fatty acid amide, or N-acyl N-alkyl derivatives of glucosamine (”glucamides“)), or a mixture thereof.

When included in the method of the invention, the concentration of the surfactant will usually be from about 0.01% to about 10%, preferably about 0.05% to about 5%, and more preferably about 0.1% to about 1% by weight.

### Methods and Uses

In a first aspect, the present invention provides an enzymatic method for killing or inhibiting growth of Mycobacteria, comprising contacting the Mycobacteria with a composition which includes a haloperoxidase, a source of hydrogen peroxide, chloride ions and/or bromide ions, and ammonium ions. In a preferred embodiment, the present invention provides a method for disinfecting or sterilizing medical devices or equipment, which comprises contacting the medical devices or equipment with the composition.

The composition may be formulated as a liquid (e.g. aqueous) or a dry product formulation. The dry product formulation may subsequently be re-hydrated to form an active liquid or semi-liquid formulation usable in the method of the invention.

When the composition is formulated as a dry formulation, the components may be mixed, arranged in discrete layers or packed separately.

In a second aspect, the invention also covers a composition which results from applying the method of the invention. In this case, the composition comprises a haloperoxidase, hydrogen peroxide, chloride ions and/or bromide ions, ammonium ions, Mycobacteria, and a medical device or equipment.

In the context of the present invention, the term “killing or inhibiting growth of Mycobacteria” is intended to mean that at least 99% of the Mycobacteria are not viable after the treatment. Preferably 99.9%, more preferably 99.99%, most preferably 99.999%, and in particular 99.9999% of the Mycobacteria are not viable.

In an embodiment, the term “disinfecting” or “disinfection” refers to high level disinfection according to “Content and Format of Premarket Notification [510(k)] Submissions for Liquid Chemical Sterilants/High Level Disinfectants”, U.S. Food and Drug Administration, January 2000.

The methods according to the invention may be carried out at a temperature between 0 and 70 degrees Celsius, preferably between 5 and 60 degrees Celsius, more preferably between 10 and 60 degrees Celsius, even more preferably between 15 and 60 degrees Celsius, even more preferably between 20 and 60 degrees Celsius, most preferably between 20 and 50 degrees Celsius, and in particular between 20 and 40 degrees Celsius.

The methods of the invention may employ a treatment time of from 10 minutes to (at least) 4 hours, preferably from 15 minutes to (at least) 3 hours, more preferably from 20 minutes to (at least) 2 hours, most preferably from 20 minutes to (at least) 1 hour, and in particular from 30 minutes to (at least) 1 hour.

The method of the invention is suitable for killing or inhibiting growth of Mycobacteria in a variety of environments. The method of the invention may desirably be used in any environment to reduce the risk of infections caused by Mycobacteria, such as in the health-care industry (e.g. animal hospitals, human hospitals, animal clinics, human clinics, dentists, nursing homes, day-care facilities for children or senior citizens, etc.), the food industry (e.g. restaurants, food-processing plants, food-storage plants, grocery stores, etc.), the hospitality industry (e.g. hotels, motels, resorts, cruise ships, etc.), the education industry (e.g. schools and universities), etc.

Due to the relatively low temperatures being utilized by the methods of the invention, they are very useful for disinfecting or sterilizing equipment, such as medical devices (e.g. dry surgical instruments, anesthesia equipment, hollowware etc), used in the health-care industry. The disinfected or sterilized equipment will exhibit reduced deformations and wear, and the equipment is ready for use substantially immediately after disinfection or sterilization. This is especially advantageous when disinfecting or sterilizing complex or heat sensitive medical devices such as ultrasound transducers and endoscopes comprising different materials, because the wear of these devices have been reduced significantly, which results in longer service life of these often very costly devices, which effectively reduces their operational cost. Indeed, even other non-medical types of equipment such as reusable hygienic articles may be disinfected or sterilized effectively by use of the present invention.

In a preferred embodiment, the disinfection or sterilization of medical devices and/or non-medical types of equipment takes place in a (Medical) Washer-Disinfector according to EN ISO 15883-1 (or as described in “Class II Special Controls Guidance Document: Medical Washers and Medical Washer-Disinfectors; Guidance for the Medical Device Industry and FDA Review Staff”, U.S. Food and Drug Administration, February 2002), using the methods of the invention.

The present invention is further described by the following examples which should not be construed as limiting the scope of the invention.

### EXAMPLES

Chemicals used as buffers and substrates were commercial products of at least reagent grade.

### Example 1

Killing of Mycobacterium bovis with haloperoxidase from Curvularia verrucolosa

The objective of this assay was to evaluate the tuberculocidal effectiveness of a product against Mycobacterium bovis—BCG following the AOAC Tuberculocidal Activity.

### Materials

Test organism: Mycobacterium bovis—BCG; obtained from Organon Teknika, Durham, USA.
Growth medium: Modified Proskauer-Beck Medium (MPB)

### Recovery Media

Neutralizer: Letheen Broth+1.0% Sodium Thiosulfate+0.01% Catalase
Subculture Medium: Modified Proskauer-Beck Medium (MPB)
   Middlebrook 7H9 Broth (7H9)
Agar Plates: Middlebrook 7H11 Agar

### Carriers

Porcelain penicylinders (O.D. 8 mm±1, I.D. 6 mm±1, length 10 mm±1) were washed with 1-5% Triton X-100 and rinsed with water at least four times, until no soap residue was present. Following washing, the carriers were macroscopically inspected for chip and cracks. Carriers with visible chips or cracks were discarded. Carriers were placed in a vessel and sterilized for 2 hours in a 180° C. air oven.

### Test Method

### Preparation of Test Substance

A total volume of 10.0 mL test substance was prepared in each 25×150 mm Morton Closure tube by mixing:

9.9 mL of solution A (125 μL of 400 mM NaCl, 250 μL of 200 mM NH₄Cl, 50 μL of 1000 mM phosphate buffer, 0.77 mg of BASF LF 900, 4475 μL MilliQ water and 5000 μL 40° dH H₂O) resulting in a final concentration of 5 mM NaCl, 5 mM NH₄Cl, 5 mM phosphate buffer and 0.077 g/L BASF LF 900;

0.05 mL of solution B (50 μL of 1600 ppm haloperoxidase from Curvularia verrucolosa (see SEQ ID NO:2 in WO 97/04102)) resulting in a final concentration of 8 ppm enzyme; and

0.05 mL of solution C (50 μL of 1600 mM H₂O₂) resulting in a final concentration of 8 mM H₂O₂.

The tubes were then placed in a 40.0° C. water bath to equilibrate for 22 minutes. Testing was performed in duplicate. The test substance was homogenous as determined by visual observation and was used within one hour of preparation.

### Preparation of Test Organism

A stock culture of the test organism, Mycobacterium bovis—BCG, was maintained on 7H11 agar medium. From the stock culture, the organism was transferred into Modified Proskauer-Beck broth and incubated for 22 days at 35-37° C. Following incubation, a 1.00 mL aliquot of 0.1% Polysorbate 80 in saline was added to the suspension. The culture broth was transferred to a sterile tissue grinder and thoroughly ground. This suspension was diluted in 10.0 mL of Modified Proskauer-Beck growth media prior to carrier contamination. The final percent transmittance of the suspension was determined to be at 9.78% T using a spectrophotometer calibrated to 650 nm.

### Contamination of Carriers

The penicylinders were immersed for 15 minutes in the ground culture at a ratio of 1 carrier per 1.0 mL culture. The carriers were then dried on filter paper in a sterile Petri dish at 35-37° C. for 30 minutes at 40% relative humidity. The drying conditions (temperature and humidity) were appropriate for the test organism for the purpose of obtaining maximum survival following drying.

### Exposure Conditions

Ten carriers per replicate of test substance were tested. Each contaminated and dried carrier was placed into a separate tube containing 10.0 mL of the test substance at its use-dilution for the 15 minute exposure time at 40.0° C.

### Test System Recovery

Each medicated carrier was transferred by wire hook at staggered intervals to 10 mL of neutralizer. The neutralized carrier was then transferred to a tube containing 20 mL Modified Proskauer-Beck subculture medium. Furthermore, a 2.0 mL aliquot of the neutralizing subculture medium was individually subcultured using 20 mL of Middlebrook 7H9 broth.

### Incubation

All broth subcultures were incubated at 35-37° C. under aerobic conditions. The subculture plates were placed in plastic bags and incubated for 15 days at 35-37° C. prior to examination. The broth subculture tubes were visually examined for growth following a 30, 60 and 90 day incubation period.

Representative subcultures demonstrating growth (≧20%) were stained using an AFB fluorescent stain to confirm identity of test organism.

### Study Controls

### Purity Control

A “streak plate for isolation” was performed on the organism culture, and following incubation, examined in order to confirm the presence of a pure culture. The acceptance criterion for this study control is a pure culture demonstrating colony morphology typical of the test organism.

### Carrier Sterility Control

A representative uninoculated carrier was added to the neutralizer. The carrier was transferred from the neutralizer to Modified Proskauer-Beck. Aliquots (2.0 mL) of the neutralizer were individually subcultured into 20 mL of Middlebrook 7H9 broth in a manner consistent with the test procedure. The subculture broths were incubated and examined for growth. The acceptance criterion for this study control is lack of growth.

### Subculture Medium Sterility Control

A representative sample of Modified Proskauer-Beck and Middlebrook 7H9 broth were incubated and visually examined. The acceptance criterion is lack of growth.

### Neutralizer Sterility Control

Aliquots (2.0 mL) of the neutralizer were added to an uninoculated representative sample of each subculture medium, Modified Proskauer-Beck and Middlebrook 7H9 broth, incubated and visually examined. The acceptance criterion is lack of growth.

### Viability Control

A representative inoculated carrier was added to the neutralizer. The carrier was then transferred to Modified Proskauer-Beck. Aliquots (2.0 mL) of the neutralizer were then subcultured to Middlebrook 7H9 broth in a manner consistent with the test procedure. The subculture broths were incubated and examined for growth. The acceptance criterion for this study control is growth.

### Neutralization Confirmation Control

The neutralization of the test substance was confirmed by exposing sterile carriers (representing not less than 10% of the total number of test carriers) to the test substance and transferring them to primary subcultures containing 10 mL of neutralizer. The carriers were subcultured to Modified Proskauer-Beck, identically to the test procedure. Aliquots (2.0 mL) of the neutralizer were then subcultured to a corresponding number of Middlebrook 7H9 broth in a manner consistent with the test procedure. The subcultures containing the exposed carriers and those to which the 2.0 mL aliquots of neutralizer had been subcultured were inoculated with low levels of test organism, incubated under test conditions and visually examined for the presence of growth. This control was performed with multiple replicates using different dilutions of the test organism. A standardized spread plate procedure was run concurrently in order to enumerate the number of CFU actually added. The control result is reported using data from the most appropriate dilution.

The acceptance criterion for this study control is growth following inoculation with low levels of test organism (≦1000 CFU) in at least one of the three types of subculture media.

### Carrier Population Control

Contaminated carriers were transferred to a sterile container of Modified Proskauer-Beck at a ratio of one carrier to 10 mL of medium and vortex mixed. This suspension was serially diluted and plated in duplicate on Middlebrook 7H11 agar plates using standard microbiological techniques. Following incubation, the organism plates were observed to enumerate the concentration of the test organism present at the time of testing. The acceptance criterion for this study control is a minimum of 1.0×10⁻⁴ CFU/carrier.

### Calculations

### Carrier Population Control Calculation:

$$C F U \text{/} carrier = \frac{\begin{matrix}( average number of colonies \text{/} plate @ dilution ) \times \\ ( dilution factor ) \times ( volume M P B )\end{matrix}}{( number of carriers tested ) \times ( volume plated )}$$

The carrier population was calculated and reported using data from the most appropriate dilution(s).

### Conditions

Exposure time: 15 minutes
Exposure temperature: 40±2° C. (40.0° C.)
Haloperoxidase concentration: 8 ppm (8 mg enzyme protein/L) and 5 mM NaCl, 5 mM NH₄Cl, 5 mM phosphate buffer, 0.077 g/L surfactant LF900, and 8 mM H₂O₂.
Test organism: Mycobacterium bovis—BCG; 1.12×10⁵ CFU/carrier (enumerated on day 15)

### Results

Viability of the test organism and sterility of the media were confirmed before carrying out the experiments (as explained above).

TABLE 1
Replicate 1.

| | | | Number of | |
|---|---|---|---|---|
| | | | carriers showing | |
| | | Total | growth of | |
| Subculture | Volume | number | Mycobacterium bovis | |
| Media | subcultured | of carriers | 30 days | 62 days |
| Modified | NA | 10 | 0 | 2 |
| Proskauer- | | | | |
| Beck | | | | |
| Middlebrook | 2.0 mL | 10 | 0 | 0 |
| 7H9 Broth | | | | |

Growth of the test organism was qualitatively observed after 30 and 62 days of incubation. After 30 days of incubation no growth was observed on any of the carriers or in the subcultured media. After 62 days of incubation 2/10 (two out of ten) carriers in modified Proskauer-Beck medium showed growth, which was validated to be the test organism Mycobacterium bovis—BCG. In the subcultured medium (Middlebrook 7H9 broth) none (0/10) of the tubes showed any growth. The haloperoxidase treatment has significantly delayed the growth of the test organism, and in 2/10 (80%) of the carriers no growth was observed.

TABLE 2
Replicate 2.

| | | | Number of carriers | | |
|---|---|---|---|---|---|
| | | Total | showing growth of | | |
| Subculture | Volume | number | Mycobacterium bovis | | |
| Media | subcultured | of carriers | 30 days | 62 days | 90 days |
| Modified | NA | 10 | 0 | 0 | 0 |
| Proskauer- | | | | | |
| Beck | | | | | |
| Middlebrook | 2.0 mL | 10 | 0 | 0 | 1 |
| 7H9 Broth | | | | | |

In replicate 2, the carriers were also observed for growth after 30, 52 and 90 days respectively. After 30 and 63 days of incubation none of the carriers or subcultured carriers showed any growth. After 90 days of incubation none of the carriers (0/10) showed any growth, whereas one (1/10) of the subcultured carriers showed growth, which was identified to be the Mycobacterium bovis—BCG.

The haloperoxidase system has in short time (15 min) at medium temperature (40° C.) at low dosage (8 ppm) significantly showed kill efficacy towards the test organism Mycobacterium bovis—BCG, as replicate 1 gave a kill efficacy of 80% and replicate 2 showed 100% kill efficacy of the test organism attached to the porcelain penicylinder carriers.

1. A method for killing or inhibiting growth of Mycobacteria, which comprises contacting the Mycobacteria with a haloperoxidase, hydrogen peroxide, chloride and/or bromide ions, and ammonium ions.

2. The method of claim 1, wherein the haloperoxidase is a chloroperoxidase from enzyme class EC 1.11.1.10.

3. The method of claim 1, wherein the haloperoxidase is a vanadium containing haloperoxidase.

4. The method of claim 1, wherein the amino acid sequence of the haloperoxidase has at least 90% identity, preferably 95% identity to the amino acid sequence of a haloperoxidase obtainable from Curvularia verruculosa (SEQ ID NO:1) or Curvularia inequalis (SEQ ID NO:2).

5. The method of claim 1, wherein the chloride ions and/or bromide ions are derived from salts of chloride and/or bromide; preferably the salts of chloride and/or bromide include sodium chloride, sodium bromide, potassium chloride, potassium bromide, ammonium chloride or ammonium bromide.

6. The method of claim 1, wherein the ammonium ions are derived from an ammonium salt; preferably the ammonium salt is ammonium sulphate, ammonium carbonate, ammonium phosphate, ammonium chloride, ammonium bromide or ammonium iodide; or a mixture thereof.

7. The method of claim 1, wherein the concentration of chloride ions is at least two times higher than the concentration of ammonium ions; preferably at least four times higher, more preferably at least six times higher, most preferably at least eight times higher, and in particular at least ten times higher than the concentration of ammonium ions.

8. The method of claim 1, which further comprises contacting the Mycobacteria with a surfactant.

9. The method of claim 1, wherein the Mycobacteria are Mycobacterium tuberculosis or Mycobacterium bovis.

10. The method of claim 1, wherein hydrogen peroxide is derived from a percarbonate salt.

11. The method of claim 1, wherein the Mycobacteria are located on a surface of a medical device or equipment.

12. The method of claim 1, which is a method of disinfection of a surface of a medical device or equipment.

13. (canceled)

14. (canceled)

15. (canceled)


SEQUENCE LISTING (2 sequences)

SEQ ID NO: 1 - protein, 600 aa, Curvularia verruculosa
MGSVTPIPLPTIDEPEEYNNNYILFWNNVGLELNRLTHTVGGPLTGPPLSARALGMLHLAIHDAYFSICPPTEFTTFLSPDAENPAYRLPSPNGADDARQAVAGAALKMLSSLYMKPADPNTGTNISDNAYAQLALVLERAVVKVPGGVDRESVSFMFGEAVADVFFALLNDPRGASQEGYQPTPGRYKFDDEPTHPVVLVPVDPNNPNGPKMPFRQYHAPFYGMTTKRFATQSEHILADPPGLRSNADETAEYDDSIRVAIAMGGAQDLNSTKRSPWQTAQGLYWAYDGSNLVGTPPRFYNQIVRRIAVTYKKEDDLANSEVNNADFARLFALVNVACTDAGIFSWKEKWEFEFWRPLSGVRDDGRPDHGDPFWLTLGAPATNTNDIPFKPPFPAYPSGHATFGGAVFQMVRRYYNGRVGTWKDDEPDNIAIDMMISEELNGVNRDLRQPYDPTAPIEDQPGIVRTRIVRHFDSAWEMMFENAISRIFLGVHWRFDAAAARDILIPTNTKDVYAVDSNGATVFQNVEDVRYSTKGTREGREGLFPIGGVPLGIEIADEIFNNGLRPTPPELQPMPQDTPVQKPVQGMWDEQVPLVKEAP

SEQ ID NO: 2 - protein, 609 aa, Curvularia inaequalis
MGSVTPIPLPKIDEPEEYNTNYILFWNHVGLELNRVTHTVGGPLTGPPLSARALGMLHLAIHDAYFSICPPTDFTTFLSPDTENAAYRLPSPNGANDARQAVAGAALKMLSSLYMKPVEQPNPNPGANISDNAYAQLGLVLDRSVLEAPGGVDRESASFMFGEDVADVFFALLNDPRGASQEGYHPTPGRYKFDDEPTHPVVLIPVDPNNPNGPKMPFRQYHAPFYGKTTKRFATQSEHFLADPPGLRSNADETAEYDDAVRVAIAMGGAQALNSTKRSPWQTAQGLYWAYDGSNLIGTPPRFYNQIVRRIAVTYKKEEDLANSEVNNADFARLFALVDVACTDAGIFSWKEKWEFEFWRPLSGVRDDGRPDHGDPFWLTLGAPATNTNDIPFKPPFPAYPSGHATFGGAVFQMVRRYYNGRVGTWKDDEPDNIAIDMMISEELNGVNRDLRQPYDPTAPIEDQPGIVRTRIVRHFDSAWELMFENAISRIFLGVHWRFDAAAARDILIPTTTKDVYAVDNNGATVFQNVEDIRYTTRGTREDPEGLFPIGGVPLGIEIADEIFNNGLKPTPPEIQPMPQETPVQKPVGQQPVKGMWEEEQAPVVKEAP